(19) United States
(12) Patent Application Publication
(10) Pub. No.: US 2023/0302009 A1
(43) Pub. Date: Sep. 28, 2023

(54) BACE1 INHIBITOR TREATMENT FOR SUPPRESSING CYTOKINE STORM

(71) Applicant: The Cleveland Clinic Foundation, Cleveland, OH (US)

(72) Inventors: Shideng Bao, Cleveland, OH (US); Kui Zhai, Beachwood, OH (US); Zhi Huang, Solon, OH (US)

(21) Appl. No.: 18/041,249

(22) PCT Filed: Aug. 9, 2021
(86) PCT No.: PCT/US21/45215

§ 371 Date: Feb. 10, 2023

Related U.S. Application Data
(60) Provisional application No. 63/064,753, filed on Aug. 12, 2020.

(51) Int. Cl. A61K 31/549; A61P 37/02; A61K 45/06; A61P 29/00; A61P 31/14

(52) U.S. Cl. CPC A61K 31/549; A61P 37/02; A61K 45/06; A61P 29/00; A61P 31/14

(57) ABSTRACT

The present invention provides compositions, systems, kits, and methods for treating a subject, having a condition that causes a cytokine storm, by administering or providing a composition comprising a beta-secretase 1 (BACE1) inhibitor. In some embodiments, the composition reduces lung or other bodily inflammation in the subject (e.g., by reducing the cytokine storm caused by a virus). In certain embodiments, the subject is infected with a virus such as SARS-CoV-2, has received CAR-T cell immunotherapy, has an organ transplant, or has an autoimmune disease (e.g., arthritis).

The present application claims priority to U.S. Provisional application Ser. No. 63/064,753 filed Aug. 12, 2020, which is herein incorporated by reference in its entirety.

### SEQUENCE LISTING

The text of the computer readable sequence listing filed herewith, titled “38717-601_SEQUENCE_LISTING_ST25”, created Aug. 9, 2021, having a file size of 4,032 bytes, is hereby incorporated by reference in its entirety.

### FIELD OF THE INVENTION

The present invention provides compositions, systems, kits, and methods for treating a subject, having a condition that causes a cytokine storm, by administering or providing a composition comprising a beta-secretase 1 (aka (3-site amyloid precursor protein cleaving enzyme 1 or BACE1) inhibitor. In some embodiments, the composition reduces lung or other bodily inflammation in the subject (e.g., by reducing the cytokine storm caused by a virus). In certain embodiments, the subject is infected with a virus such as SARS-CoV-2, has received CAR-T cell immunotherapy, has an organ transplant, or has an autoimmune disease (e.g., arthritis).

### BACKGROUND

The outbreak of COVID-19 caused by SARS-CoV-2 coronavirus has rapidly become a global pandemic, resulting in more than 20 million confirmed cases with over 740,000 deaths worldwide so far. Approximately 20% of COVID-19 patients develop severe symptoms manifested by the overwhelming inflammation, pneumonia, high fever and lung damage, leading to acute respiratory distress syndrome (ARDS) (1-4). Recent reports showed that some severe patients with COVID-19 also develop abnormal coagulation including unusual blood clotting in the lung and other organs (5-8). These severe symptoms are mainly caused by the excess release of pro-inflammatory cytokines/chemokines called cytokine storm or cytokine release syndrome (CRS) after the viral infection (2, 9-11). The cytokine storm-induced ARDS was also observed in severe patients with SARS or MERS and in leukemia patients treated with CAR-T immunotherapy (12-14). Thus, cytokine storm represents an excessively exaggerated immune response most often accompanying selected viral infections caused by certain virus such as SARS-CoV, MERS-CoV, influenza and SARS-CoV-2. The mortalities of these viral infections are often the direct results of cytokine storm that triggers hyper-inflammation to cause tissue damage and organ failure (15-17). Severe patients with COVID-19 usually have markedly high plasma levels of several pro-inflammatory cytokines/chemokines such as IL-6, IL-1β, TNF-α, G-CSF, GM-CSF, IL-10, IL-2, IP10, MCP1 and others (2, 18). Therefore, overcoming the cytokine storm to inhibit the overwhelming inflammation is important for relieving severe symptoms including the ARDS to improve the survival of severe patients with COVID-19. Therapeutics that can overall suppress the cytokine storm to relive the inflammation-related symptoms are urgently needed to improve COVID-19 treatment and significantly reduce death of severe patients with COVID-19.

### SUMMARY OF THE INVENTION

The present invention provides compositions, systems, kits, and methods for treating a subject, having a condition that causes a cytokine storm, by administering or providing a composition comprising a beta-secretase 1 (BACE1) inhibitor. In some embodiments, the composition reduces lung or other bodily inflammation in the subject (e.g., by reducing the cytokine storm caused by a virus). In certain embodiments, the subject is infected with a virus such as SARS-CoV-2, has received CAR-T cell immunotherapy, has an organ transplant, or has an autoimmune disease (e.g., arthritis).

In some embodiments, provided herein are methods of treating a subject with a condition that causes a cytokine storm in the subject comprising: administering a composition to a subject, or providing the composition to the subject such that the subject administers the composition to themselves, wherein the subject has a condition that causes a cytokine storm, and wherein the composition comprises a beta-secretase 1 (BACE1) inhibitor.

In certain embodiments, provided herein are compositions comprising: a) a physiologically tolerable buffer; b) a BACE1 inhibitor; and c) an anti-inflammatory agent and/or an agent that reduces SARS-CoV-2 (or other respiratory virus) replication or infection rate in vivo.

In further embodiments, provided herein are kits or systems comprising: a) a BACE1 inhibitor; and b) an anti-inflammatory agent and/or an agent that reduces SARS-CoV-2 (or other respiratory virus) replication or infection rate in vivo.

In particular embodiments, the condition is infection by a virus that causes the cytokine storm. In further embodiments, the virus is SARS-CoV-2 causing COVID-19. In other embodiments, the virus is a coronavirus. In some embodiments, the virus is selected from the group consisting of: MERS, SARS-COV-1, influenza, RSV, adenovirus, and Ebola. In certain embodiments, the condition is: having received CAR-T cell immunotherapy, having receive an organ transplant, and/or having an autoimmune disease. In further embodiments, the autoimmune disease is arthritis.

In particular embodiments, the providing comprises giving the composition to the subject in the form of oral pills that the patient takes themselves. In other embodiments, the administering comprises injecting the composition into the subject. In additional embodiments, the methods further comprise: c) repeating the administering or providing daily for at least one week or at least three weeks. In certain embodiments, the composition further comprises a physiologically tolerable buffer.

In some embodiments, the BACE1 inhibitor comprises MK-8931 (Verubecestat). In other embodiments, the BACE1 inhibitor is selected from the group consisting of: AZD3293 (Lanabecestat), E2609 (Elenbecestat), CNP50, CNP2609, PF-06751979, and JNJ-54861911 (Atabecestat). In further embodiments, the BACE1 inhibitor is selected from Table 1.

In particular embodiments, the subject is a human. In certain embodiments, the administering comprises intravenous administration. In additional embodiments, the administering is via the subject's airway. In additional embodiments, the composition is freeze-dried and administered via the subject's airway, or provided in a nebulizer. In other embodiments, the methods further comprise: administering or providing an anti-coagulant to the subject. In some embodiments, the methods further comprise: administering or providing an anti-viral agent to the subject. In additional embodiments, the antiviral agent comprises Remdesivir or an anti-SARS-CoV-2 antibody or fragment thereof. In further embodiments, the anti-viral agent is such that it reduces SARS-CoV-2 replication or infection rate in vivo.

In certain embodiments, the methods further comprise: administering an anti-inflammatory agent to the subject. In some embodiments, the anti-inflammatory agent comprises dexamethasone. In other embodiments, the subject has lung inflammation. In particular embodiments, the subject is on a ventilator. In additional embodiments, the subject has general body inflammation.

In some embodiments, the administering comprises administering 0.05 mg of the BACE1 inhibitor per kg of the subject to 50 mg of the BACE1 inhibitor per kg of the subject (e.g., 0.05 . . . 1.0 . . . 10 . . . 30 . . . or 50 mg/kg), or administering a total dose of 3-1000 mg of the BACE1 inhibitor (e.g., 3 . . . 100 . . . 400 . . . 800 . . . 1000 mg). In other embodiments, the administering is such that the subject receives about 0.5-4.0 mg of the BACE1 inhibitor per kilogram of the patient 1-5 times per day for at least 1 day (e.g., at least 1 day . . . 3 days . . . 10 days . . . or 30 days). In some embodiments, the methods further comprise: repeating the administering daily for at least one week or at least three weeks (e.g., at least 7 . . . 14 . . . 21 . . . 28 . . . 35 . . . or 100 days).

### DESCRIPTION OF THE FIGURES

FIG. 1. Spike protein of SARS-CoV-2 activates macrophages to release cytokines and chemokines. (A) A brief protocol for generating monocytes and macrophages from human iPS cells (iPSCs) expressing GFP. Human iPSCs (GFP+) were seeded on an ultra-low attachment plate and cultured in the iPSC medium supplemented with BMP4 (50 ng/mL), SCF (20 ng/mL), VEGF (20 ng/mL), and Y27632 (50 μM) for four days to form embryoid bodies (EBs). Then, the EBs were incubated in the X-VIVO medium with IL3 (25 ng/mL) and M-CSF (100 ng/mL) for two weeks to generate monocytes. Finally, the iPCS-derived monocytes were collected and induced to differentiate into macrophages by M-CSF (100 ng/mL) in the X-VIVO medium. (B) Immunofluorescent analysis of macrophage markers in the iPSC-derived macrophages. The iPSC-derived macrophages (GFP+) and the matched iPSCs (GFP+, control) were stained with specific antibodies against the macrophage markers CD11b or IBA1 (in red) and then counterstained with DAPI (in blue). Representative immunofluorescent images showing that both CD11b and IBA1 were expressed by the iPSC-derived macrophages but not by the iPSCs. Scale bars represent 30 μM. (C and D) Immunofluorescent analysis of angiotensin-converting enzyme 2 (ACE2) expression in iPSC- or U937-derived macrophages. The iPSC-derived macrophages (C) or U937-derived macrophages (D) were stained with the specific antibody against ACE2 (in red for iPSC-derived macrophages or in green for U937-derived macrophages), and then counterstained with DAPI (in blue). Representative immunofluorescent images showing ACE2 expression by the iPSC- and U937-derived macrophages. Scale bars represent 30 μM. (E) Immunoblot analysis of ACE2 expression in U937 monocytes, iPSC-derived macrophages, and A549 lung cells. A549 cells were used as a positive control for ACE2 expression. GAPDH was blotted as the loading control. Representative immuno-blot shows ACE2 expression by both U937 monocytes and iPSC-derived macrophages. (F and G) Cytokine secretion profile in the conditioned media of U937-derived macrophages stimulated by the SARS-Cov-2 Spike protein or the control (BSA). U937-derived macrophages were incubated with the recombinant Spike protein of SARS-CoV-2 (10 μg/mL) or BSA control (10 μg/mL) in the serum-free RPMI-1640 medium for 24 h. Conditioned media were harvested and concentrated by the trichloroacetic acid (TCA) method. The cytokine secretion profiles in the conditioned media were determined by using the Proteome Profiler Human Cytokine Array Kit. Representative images of cytokine array shows several cytokines or chemokines (boxed) were induced by SARS-Cov-2 spike protein in macrophages (F). The individual dots corresponding to those specific cytokines/chemokines (1-9) highly induced by the stimulation of SARS-Cov-2 Spike protein in macrophages were shown and indicated (G). (H) Quantified analysis of the above cytokine array by Image J showing the relative expression levels of those specific cytokines/chemokines induced by the SARS-Cov-2 Spike protein in macrophages. The levels of these cytokines/chemokines in the conditioned media from the BSA-treated macrophages were set to 1, so the increased folds of cytokines/chemokines induced by the Spike protein were directly indicated. Data are shown as mean±SD (n=4). **p<0.01 and ***p<0.001; Student's t test.

FIG. 2. BACE1 inhibition by MK-8931 suppresses the SARS-CoV-2 Spike protein-induced cytokine release by macrophages. (A and B) Cytokine secretion profile in the conditioned media of U937-derived macrophages treated with the BSA control, SARS-CoV-2 Spike protein, or MK-8931 plus the Spike protein. U937-derived macrophages were incubated with BSA control (10 μg/mL), the recombinant Spike protein of SARS-CoV-2 (10 μg/mL), or MK-8931 (50 μg/mL) plus the Spike protein (10 μg/mL) in the serum-free RPMI-1640 medium for 24 h. Conditioned media were harvested and concentrated by the trichloroacetic acid (TCA) method. The cytokine secretion profiles in the conditioned media were determined by using the Proteome Profiler Human Cytokine Array Kit. Representative images of cytokine array shows several cytokines or chemokines (boxed) were induced by SARS-Cov-2 Spike protein in macrophages, but such inductions were abolished by MK-8931 treatment (A). The individual dots corresponding to those specific cytokines/chemokines (1-9) highly induced by the stimulation of SARS-Cov-2 Spike protein but suppressed by MK-8931 co-treatment in macrophages were shown and indicated (B). (C) Quantified analysis of the cytokine array by Image J showing the relative expression levels of those specific cytokines/chemokines induced by the SARS-Cov-2 Spike protein but suppressed by MK-8931 co-treatment in macrophages. The levels of these cytokines/chemokines in the conditioned media from the BSA-treated macrophages were set to 1, so the relative changes of cytokines/chemokines induced by the Spike protein (S protein) and then suppressed by MK-8931 co-treatment were indicated. Data are shown as mean±SD (n=4). **p<0.01 and ***p<0.001; Student's t test. (D) RT-qPCR analysis showing the relative mRNA expression levels of the specific cytokines/chemokines induced by SARS-CoV-2 Spike (S) protein and then suppressed by MK-8931 co-treatment. The mRNA levels of the cytokines/chemokines in the BSA-treated macrophages were set to 1, so the relative mRNA levels of cytokines/chemokines induced by the Spike protein (S protein) and then suppressed by MK-8931 co-treatment were indicated. Data are shown as mean±SD (n=3). *p<0.05, **p<0.01 and ***p<0.001; Student's t test.

FIG. 3. MK-8931 treatment effectively attenuates the LPS-induced cytokine storm in vivo. (A and B) Plasma cytokine profile in serum from mice treated with DMSO (Control), MK-8931, LPS, or LPS plus MK-8931. To induce cytokine storm in vivo, mice were intraperitoneally injected with a single dose of LPS (15 mg/kg). MK-8931 (50 mg/kg) or the vehicle control was orally administered into mice after the LPS treatment for 1 and 13 hours. Twenty-four hours after LPS administration, four groups of mice were anesthetized and plasma samples were harvested for the cytokine array. Representative images of cytokine array shows a number of cytokines or chemokines (boxed) were induced by LPS stimulation in vivo, but MK-8931 treatment attenuated such induction (A). The individual dots corresponding to these specific cytokines or chemokines (1-20) highly induced by the LPS stimulation but suppressed by MK-8931 co-treatment in vivo were shown and indicated (B). Those cytokines or chemokines labeled in red color were reported to be also increased in COVID-19 patients, and the cytokines or chemokines labeled in blue color were also induced by the SARS-Cov-2 Spike protein in macrophages in vitro. (C) Quantified analysis showing the relative plasma levels of the specific cytokines or chemokines in the serum from mice treated with LPS, LPS plus MK-8931 or the vehicle control. The levels of the cytokines/chemokines in the serum from the control mice were set to 1, so the relative changes of cytokines/chemokines induced by LPS and then suppressed by MK-8931 co-treatment were indicated. Data are shown as mean±SD (n=4). *p<0.05, **p<0.01, and ***p<0.001 indicate the significances between the control and LPS-treated mice; #p<0.05, ##p<0.01, and ###p<0.001 indicate the significances between the LPS-treated mice and the LPS plus MK-8931-treated mice; Student's t test.

FIG. 4. MK-8931 treatment effectively protects animals from the cytokine storm-induced death caused by lethal dose of LPS. (A) A schematic schedule showing the preclinical trial of MK-8931 treatment to rescue the cytokine storm-induced death caused by lethal dose of LPS. Briefly, a single dose of LPS (15 mg/kg) was intraperitoneally injected into two groups of mice (14 mice/group). MK-8931 (50 mg/kg) or the vehicle control (DMSO) was orally administered into the experimental or control group of mice before and after LPS treatment at −24, 0, 12, 24, 48, and 72 hours. Then two groups of mice were closely monitored to record the survival or death within seven days. (B) Kaplan-Meier survival curves of LPS-challenged mice treated with MK-8931 or the vehicle control (DMSO). 14 mice were included in each group. Log-rank analysis was used to assess the significance. MK-8931 treatment significantly protected animals from the cytokine storm-induced death caused by lethal dose of LPS. **p<0.01 as indicated. (C and D) Histological analysis showing MK-8931 treatment remarkably attenuated the LPS-induced injury in the lung and kidney. Representative hematoxylin and eosin (HE)-stained images of lung (C) and kidney (D) tissues are shown. Quantifications show the tissue injury scores in the lung and kidney from mice treated with Control, MK-8931, LPS only or LPS plus MK-8931. Scale bars represent 50 μM. Data are shown as mean±SD (n=6 mice). ***p<0.001; Student's t test. (E and F) Histological analysis of the spleen and liver tissues from mice treated with LPS only or LPS plus MK-8931. Representative HE-stained images of spleen (E) and liver (F) tissues are shown. Quantifications show the tissue injury scores in the spleen and liver from mice treated with Control, MK-8931, LPS only or LPS plus MK-8931. No significant tissue injury was found in the spleen and liver from mice treated with LPS only or LPS plus MK-8931. Scale bars represent 50 μM. Data are shown as mean±SD (n=6 mice).

FIG. 5. A Schematic Illustration shows MK-8931 treatment suppresses the cytokine storm induced by SARS-CoV2 spike protein or LPS. Cytokine storm and extensive macrophage infiltration are commonly observed in the severe COVID-19 patients that have been found to the major causes of mortality. In was found that the spike protein of SARS-CoV2 is sufficient to induce the cytokine storm in macrophages characterized by the secretion of multiple cytokines and chemokines. This is perhaps due to the binding of the spike protein of SARS-Cov-2 with angiotensin-converting enzyme 2 (ACE2) which is shown to be expressed by macrophages. Of clinical significance, the cytokine storm induced by the SARS-Cov-2 spike protein in macrophages is almost entirely blocked by MK-8931 treatment. By using a LPS-induced cytokine storm model, we further demonstrated that MK-8931 treatment significantly blocked the LPS-induced cytokine storms both in vitro and in vitro and protected the mice from LPS-induced tissue damages and death.

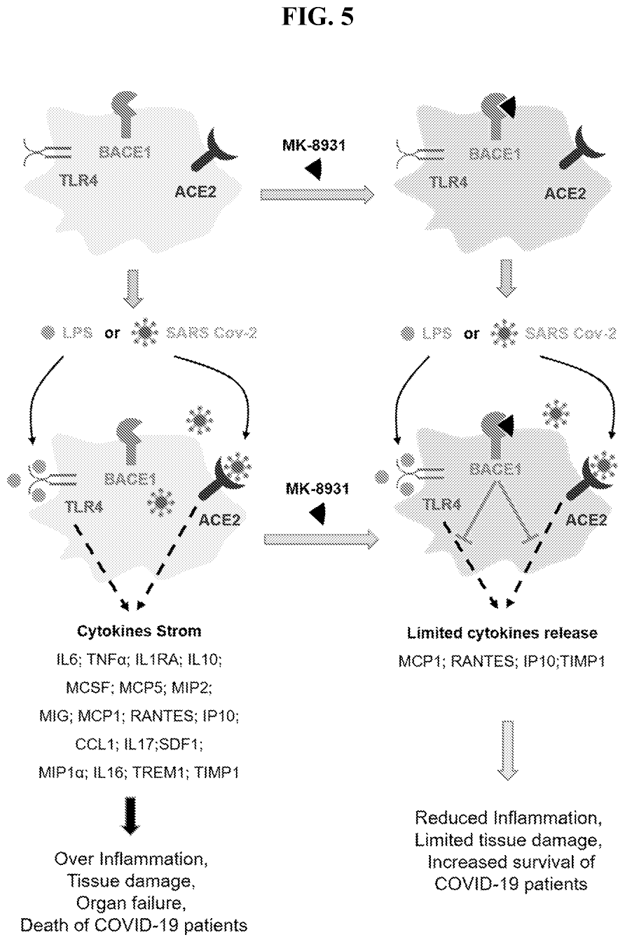

FIG. 6. BACE1 is expressed by the iPSC-derived macrophages and U937 monocyte-derived macrophages. Immunofluorescent staining of BACE1 in iPSC-derived macrophages (A) and U937 monocyte-derived macrophages (B). iPSC-derived macrophages and U937-derived macrophages were immunostained with a specific antibody against BACE1 (in red or green) and counterstained with DAPI for detecting nuclei (in blue).

FIG. 7. BACE1 inhibition by MK-8931 suppresses the LPS-induced cytokine release by macrophages. (A and B) Cytokine secretion profile in the conditioned media of U937-derived macrophages treated with LPS (lipopolysaccharide), LPS plus MK-8931, or the vehicle control. U937-derived macrophages were incubated with the vehicle control (DMSO), LPS (1 μg/mL), or MK-8931 (50 μg/mL) plus LPS (1 μg/mL) in the serum-free RPMI-1640 medium for 24 h. Conditioned media were harvested and concentrated by the trichloroacetic acid (TCA) method. The cytokine secretion profiles in the conditioned media were determined by using the Proteome Profiler Human Cytokine Array Kit. Representative images of cytokine array (A) shows several cytokines or chemokines (boxed) were induced by LPS stimulation in macrophages, but such inductions were abolished by MK-8931 treatment. The individual dots corresponding to those specific cytokines/chemokines (1-7) highly induced by the LPS stimulation but suppressed by MK-8931 co-treatment in macrophages were shown and indicated (B). Several key cytokines/chemokines including G-CSF, GM-CSF, TNF-a, IL-1b, IL-6, IL-10, and Serpin E1 were markedly induced by LPS stimulation, but such inductions were attenuated by MK-8931 co-treatment. (C) Quantified analysis of the above cytokine array by Image J showing the relative expression levels of those specific cytokines/chemokines induced by LPS stimulation but suppressed by MK-8931 co-treatment in macrophages. The levels of these cytokines/chemokines in the conditioned media from the control macrophages were set to 1, so the relative changes of cytokines/chemokines induced by LPS stimulation and then suppressed by MK-8931 co-treatment were indicated. Data are shown as mean±SD (n=4). **p<0.01 and ***p<0.001; Student's t test. (D) RT-qPCR analysis showing the relative mRNA expression levels of the specific cytokines/chemokines induced by LPS stimulation and then suppressed by MK-8931 co-treatment. The mRNA levels of the cytokines/chemokines in the BSA-treated macrophages were set to 1, so the relative mRNA levels of cytokines/chemokines induced by LPS stimulation and then suppressed by MK-8931 co-treatment were indicated. Data are shown as mean±SD (n=3). **p<0.01 and ***p<0.001; Student's t test.

### DETAILED DESCRIPTION

The present invention provides compositions, systems, kits, and methods for treating a subject, having a condition that causes a cytokine storm, by administering or providing a composition comprising a beta-secretase 1 (BACE1) inhibitor. In some embodiments, the composition reduces lung or other bodily inflammation in the subject (e.g., by reducing the cytokine storm caused by a virus). In certain embodiments, the subject is infected with a virus such as SARS-CoV-2, has received CAR-T cell immunotherapy, has an organ transplant, or has an autoimmune disease (e.g., arthritis).

Cytokines are mainly secreted by immune cells, particularly macrophages and monocytes. Activated macrophages are the major cell source releasing the pro-inflammatory cytokines and key mediators of the immunopathology (19, 20). Several critical cytokines including IL-6, G-CSF, IL-1β, TNF-α, IL-10, and GM-CSF are expressed and secreted by macrophages and monocytes in response to the viral or bacterial infection. Consistently, accumulations of macrophages in lungs of COVID-19 patients have been reported (21, 22). Autopsy of patients who died of COVID-19 showed that the major infiltrated immune cells in lung alveoli were macrophages and monocytes (11), indicating that monocyte- and macrophage-secreted pro-inflammatory cytokines/chemokines are the major factors to induce hyper-inflammation in the lung after SARS-CoV-2 infection. SARS-CoV-2 gain entry into cells mainly through the binding of its Spike protein to ACE2 receptor on host cells (23, 24). As ACE2 is not only expressed on alveolar type II pneumocytes in cardiopulmonary tissues but also expressed by some hematopoietic cells, particularly macrophages and monocytes (25), SARS-CoV-2 as a member of the betacoronavirus family likely activates macrophages and monocytes to induce release of pro-inflammatory cytokines (18, 26). Because excessive cytokine release by dysregulated macrophages contributes to the development of life-threatening symptoms in severe patients with COVID-19, therapeutics that can regulate macrophage function to reduce the production and secretion of pro-inflammatory cytokines is urgently needed. In word conducted during the development of embodiments herein, it was found that manipulating macrophages by the BACE1 inhibitor MK-8931 suppresses the SARS-CoV-2-induced release of several pro-inflammatory cytokines/chemokines simultaneously. Thus, BACE1 inhibitors could be employed for COVID-19 treatment (e.g., to improve the survival of severe patients with COVID-19).

In certain embodiments, the BACE1 inhibitor employed herein is provided in Table 1.

TABLE 1

| BACE1 inhibitor | Company | Structure |
|---|---|---|
| MK-8931/ Verubecestat | Merck & Co. | 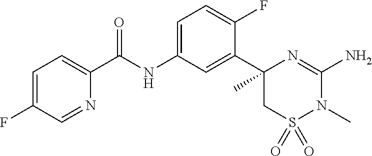 [SMILES: CN1C(N)=N[C@](C)(c2cc(NC(=O)c3ccc(F)cn3)ccc2F)CS1(=O)=O] |
| AZD3293/ LY3314814/ Lanabecestat | AstraZeneca | 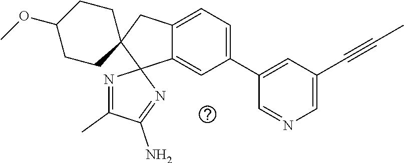 |
| AZD3839 | AstraZeneca | 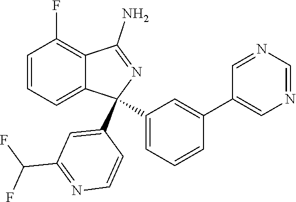 [SMILES: NC1=N[C@@](c2cccc(-c3cncnc3)c2)(c2ccnc(C(F)F)c2)c2cccc(F)c21] |
| E2609/ Elenbecestat | Eisai/Biogen | 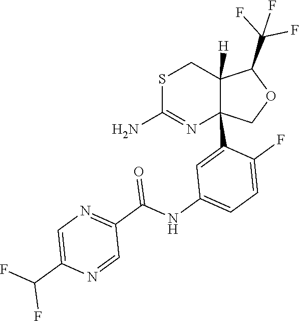 [SMILES: NC1=N[C@@]2(c3cc(NC(=O)c4cnc(C(F)F)cn4)ccc3F)CO[C@H](C(F)(F)F)[C@H]2CS1] |
| CNP520/ Umibecestat | Novartis/Amgen | 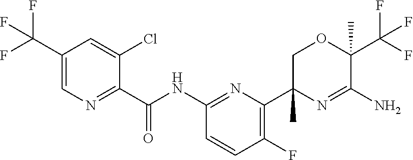 [SMILES: C[C@@]1(c2nc(NC(=O)c3ncc(C(F)(F)F)cc3Cl)ccc2F)CO[C@@](C)(C(F)(F)F)C(N)=N1] |
| JNJ-54861911/ Atabecestat | Janssen/Shionogi | 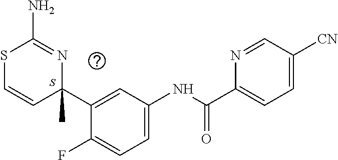 |
| PF-06751979 | Pfizer | 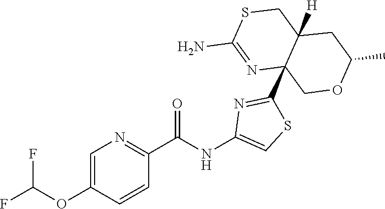 [SMILES: C[C@H]1C[C@H]2CSC(N)=N[C@@]2(c2nc(NC(=O)c3ccc(OC(F)F)cn3)cs2)CO1] |
| CTS-21166 | CoMentis | 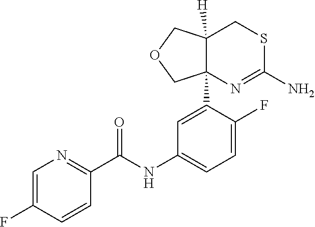 [SMILES: NC1=N[C@@]2(c3cc(NC(=O)c4ccc(F)cn4)ccc3F)COC[C@H]2CS1] |
| HPP854 | High Point | 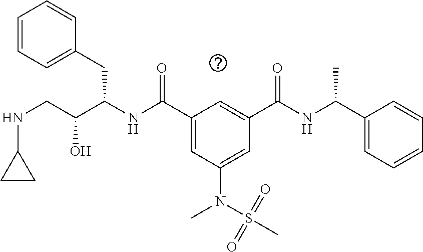 [SMILES: C[C@@H](NC(=O)c1cc(C(=O)N[C@@H](Cc2ccccc2)[C@H](O)CNC2CC2)cc(N(C)S(C)(=O)=O)c1)c1ccccc1] |
| TAK-070 | Takeda | 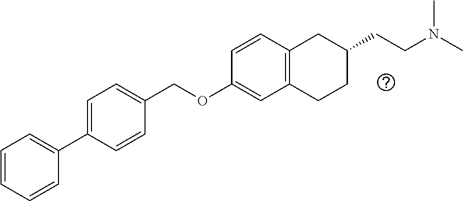 [SMILES: CN(C)CC[C@@H]1CCc2cc(OCc3ccc(-c4ccccc4)cc3)ccc2C1] |
| VTP-37948 | Vitae/Boehringer Ingelheim | 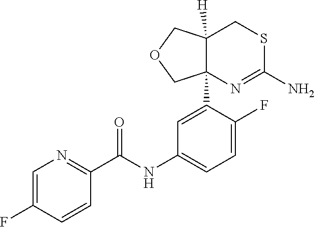 [SMILES: NC1=N[C@@]2(c3cc(NC(=O)c4ccc(F)cn4)ccc3F)COC[C@H]2CS1] |
| LY2886721 | Lilly | 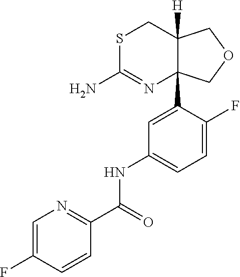 [SMILES: NC1=N[C@@]2(c3cc(NC(=O)c4ccc(F)cn4)ccc3F)COC[C@H]2CS1] |
| PF-05297909 | Pfizer | 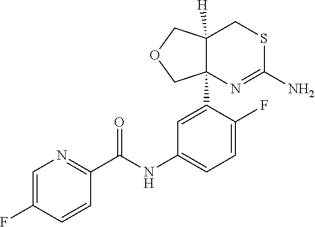 [SMILES: NC1=N[C@@]2(c3cc(NC(=O)c4ccc(F)cn4)ccc3F)COC[C@H]2CS1] |
| RG7129 | Roche | 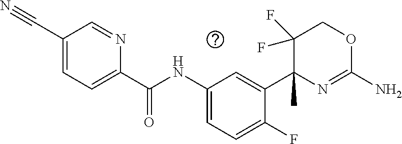 |

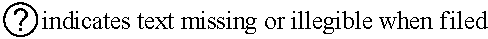

In certain embodiments, the BACE1 inhibitor employed herein is any of the compounds in Hsiao et al., Bioorganic & Medicinal Chemistry Letters 29 (2019) 761-777, including any of compounds 1-104. Hsiao et al. is incorporated by reference in its entirety, and specifically for any of the compounds recited therein. In other embodiments, the BACE1 inhibitor is any of the compounds in Moussa-Pacha et al., Med Res Rev. 2019; 1-46, including any of the compounds in Table 2. Moussa-Pacha et al. is incorporated by reference in its entirety, and specifically for the compounds listed in Table 2.

### EXAMPLES

### Example 1

### BACE1 Inhibitor Treatment

COVID-19 rapidly emerges as a global pandemic causing high mortality. The death by COVID-19 is mainly caused by SARS-CoV-2-induced cytokine storm that triggers hyper-inflammation and severe symptoms including ARDS. Thus, overcoming cytokine storm is critical for improving survival of COVID-19 patients. Here, we report that BACE1 inhibitor potently suppresses cytokine release induced by SARS-CoV-2 Spike protein or LPS and prevents cytokine storm-related death. SARS-CoV-2 Spike protein induces expression and secretion of several pro-inflammatory cytokines/chemokines by macrophages, but BACE1 inhibition by MK-8931 effectively attenuates such induction. MK-8931 treatment also abolishes the LPS-induced cytokine storm. Importantly, MK-8931 treatment protects animals from the cytokine storm-induced death caused by lethal dose of LPS. These findings indicate that treatment with the BACE1 inhibitor should improve survival of severe COVID-19 patients.

### Materials and Methods

### Human iPSC-Derived Monocytes and Macrophages

Human iPS cells (iPS11) were obtained from ALSTEM and grown in the mTeSR1 medium (StemCell Technologies, 85850). Human iPSC-derived monocytes and macrophages were prepared according to an established protocol (36-38). Briefly, human iPS cells were seeded on an ultra-low attachment plate (Costar, 7007) in 100 μL of the mTeSRTM1 medium supplemented with BMP4 (50 ng/mL, Abcam, ab87063), SCF (20 ng/mL, Peprotech, 300-07), VEGF (20 ng/mL, Peprotech, 100-20), and Y27632 (50 μM, SellckChem, S1049) to induce the formation of embryoid bodies (EBs). The 96-well ultra-low attachment plate was centrifuged at 800 rpm for three minutes and the plate was placed into the incubator for four days. At day 2, 50 μL of culture medium in the well was replaced with 50 μL of fresh mTeSRTM1 medium containing the above inducers. For monocyte differentiation, around ten EBs were transferred into each well of a six-well plate and cultured in the X-VIVOTM 15 medium (Lonza, 04-418Q) supplemented with IL3 (25 ng/mL, Biolegend, 578006), M-CSF (100 ng/mL, Biolegend, 574806), glutamine (2 mM, ThermoFisher, 35050061), and β-mercaptoethanol (0.055 M, ThermoFisher, 21985023) for two weeks. The medium was changed every 5 days. Once monocytes were visible in the supernatant of the cultures, non-adherent monocytes were harvested. To generate macrophages derived from the monocytes, the iPSC-derived monocytes (1.5×105) were plated on each well of 6-well plates and cultured in the X-VIVOTM 15 medium with M-CSF (100 ng/mL, Biolegend, 574806) for six days.

### U937 Monocyte-Derived Macrophages

The human monocyte cell line U937 was obtained from the American Type Culture Collection (ATCC; CRL-1593.2) and maintained at 37° C. in a humidified incubator with 5% CO2 and 95% air atmosphere. U937 cells were cultured in the RPMI-1640 supplemented with 10% FBS (Fetal Bovine Serum; Gibco) and 1% penicillin-streptomycin and were consistently confirmed to be free from mycoplasma by using a MycoFluor™ Mycoplasma Detection Kit (ThermoFisher, M7006). The U937-derived macrophages were prepared according to an established protocol (37, 38). In brief, U937 cells grown in a 10-cm tissue culture dish were primed by PMA (5 nM) for 48 hours, and then induced by IL4 (20 ng/mL, Peprotech, 200-04), IL10 (20 ng/mL, Peprotech, 200-10), and TGFβ (20 ng/mL, Peprotech, 200-21) for 72 hours to generate macrophages.

### Chemical and Reagents

Recombinant human coronavirus SARS-CoV-2 Spike Glycoprotein S1 was purchased from Abcam (ab272105). MK-8931 was purchased from Selleckchem (S8173). Paraformaldehyde (PFA) was obtained from Electron Microscopy Sciences (15714). Protease (04693159001) and phosphatase inhibitors (04906837001) tablets were from Roche. Recombinant Human IL4 (200-04), IL10 (200-10), and TGFβ (200-21) were obtained from Peprotech. The other chemicals and reagents otherwise indicated were from Sigma-Aldrich.

### Conditioned Medium and Cytokine Array

To profile cytokines/chemokines in conditioned media of macrophages through cytokine array, the U937 monocyte- or iPSC-derived macrophages were washed twice with PBS and then cultured in the RPMI-1640 medium in the presence or absence of SARS-CoV-2 Spike Glycoprotein S1 (50 μg/mL) or lipopolysaccharides (LPS; 1 μg/mL) and in combination with or without MK-8931 treatment (50 μg/mL) for 24 hours, and the conditioned media were harvested and concentrated by the trichloroacetic acid (TCA) method. To detect and profile cytokines/chemokines in the conditioned media of macrophages with different treatment, the human cytokine array was performed by using the Proteome Profiler Human Cytokine Array Kit (R&D, ARY005B) according to the manufacturer's instruction. Briefly, conditioned media from different treatment groups of macrophages were mixed with a cocktail of biotinylated human cytokine detection antibodies and incubated at room temperature for 1 hour. Then, the mixtures of cytokines and detected antibodies were incubated with the Human Cytokine Array membrane at 4° C. overnight. The membranes were washed three times and then incubated with the diluted Streptavidin-HRP buffer for 30 minutes at room temperature. After washing, immunoreactivity was visualized using the chemiluminescence reagent mix, and the signals were acquired and analyzed by Image Lab software (Bio-Rad). The intensity was measured and quantified by using image J software (NIH, Bethesda, MD, USA).

### RNA Isolation and qRT-PCR

Total RNA was extracted with the PureLink™ RNA Kit (ThermoFisher, 12183020) and cDNA were produced by using the M-MLV reverse transcriptase (Promega, PR-M1701). Real-time PCR (qPCR) was carried out on an ABI 7500 Real-Time PCR System (Applied Biosystems) using the SYBR-green qPCR Kit (Alkali Scientific, QS2050). Expression values were normalized to GAPDH. Gene-specific primers were as follows: G-CSF (Forward, F): 5′-AAT CAT GGA GGA GGA TGC CTT-3′ (SEQ ID NO:1), G-CSF (Reverse, R): 5′-GTC ACA GCG GAG ATA GTG CC-3′ (SEQ ID NO:2); GM-CSF (F): 5′-GCA TGT GAA TGC CAT CCA GG-3′ (SEQ ID NO:3), GM-CSF (R): 5′-CAC AGG AAG TTT CCG GGG TT-3′ (SEQ ID NO:4); ICAM1 (F): 5′-AGA GTT GCA ACC TCA GCC TC-3′ (SEQ ID NO:5), ICAM1 (R): 5′-AAC AAC TTG GGC TGG TCA CA-3′ (SEQ ID NO:6); IL10 (F): 5′-GCC TAA CAT GCT TCG AGA TC-3′ (SEQ ID NO:7), IL10 (R): 5′-TGA TGT CTG GGT CTT GGT TC-3′ (SEQ ID NO:8); SERPINE1 (F): 5′-GGA GAA ACC CAG CAG CAG AT-3′ (SEQ ID NO:9), SERPINE1 (R): 5′-CCG GAA CAG CCT GAA GAA GT-3′ (SEQ ID NO:10); IL1B (F): 5′-TTT GAG TCT GCC CAG TTC CC-3′ (SEQ ID NO:11), IL1B (R): 5′-TCA GTT ATA TCC TGG CCG CC-3′ (SEQ ID NO:12); IL6 (F): 5′-TGA CAA ACA AAT TCG GTA CAT CCT-3′ (SEQ ID NO:13), IL6 (R): 5′-AGT GCC TCT TTG CTG CTT TCAC-3′ (SEQ ID NO:14); TNFA (F): 5′-GAG CAC TGA AAG CAT GAT CC-3′ (SEQ ID NO:15), TNFA (R): 5′-CGA GAA GAT GAT CTG ACT GCC-3′ (SEQ ID NO:16); MIF (F): 5′-AGA ACC GCT CCT ACA GCA AG-3′ (SEQ ID NO:17), MIF (R): 5′-GCG AAG GTG GAG TTG TTC CA-3′ (SEQ ID NO:18); and GAPDH (F): 5′-AAG GTG AAG GTC GGA GTC AA C-3′ (SEQ ID NO:19), GAPDH (R) 5′-GGG GTC ATT GAT GGC AAC AAT A-3′ (SEQ ID NO:20).

### Immunoblot and Immunofluorescence Analysis

Immunoblot analysis was performed as previously reported (38-44). In brief, cells were lysed in RIPA buffer containing protease and phosphatase inhibitors (Roche) for 20 min on ice. After incubation in RIPA, the lysates were centrifuged at 12,000 rpm for 15 min and the supernatant was collected. Protein concentration was determined by using the Bradford assay (Bio-Rad Rad Laboratories). Precleared protein samples were subject to SDS-PAGE and blotted onto PVDF membranes. After blockade by 5% non-fat dry milk in TBST, blots were incubated with primary antibodies overnight at 4° C. followed by HRP-linked species-specific antibodies (Santa-Cruz). The following antibodies were used for immunoblot: anti-BACE1 (Santa Cruz, sc-33711, 1:200), anti-ACE2 (R&D, AF933, 1:1000), and anti-GAPDH (Cell signaling, 2118, 1:3000). The membranes were washed three times in the TBST buffer (five minutes each) and then soaked in the 5% non-fat dry milk TBST buffer containing the diluted HRP-labeled secondary antibodies for one hour at room temperature. After three washes of TBST, immunoreactivity was visualized using the chemiluminescence reagent mix and the signals were acquired and analyzed by Image Lab software (Bio-Rad).

Immunofluorescent staining was performed as described previously (38-40, 42, 44). Cells were fixed with 4% PFA for ten minutes, washed three times by cold PBS for five minutes, and blocked with 3% (w/v) BSA (Sigma-Aldrich, A7906) in PBS for one hour at room temperature. Primary antibodies were added to the cells and incubated overnight at 4° C. The following primary antibodies were used or immunofluorescence: anti-BACE1 (Thermo Fisher Scientific, MA1-177, 1:50) and anti-ACE2 (R&D, AF933, 1:50). After the incubation of the primary antibodies, the cells were washed three times by cold PBS and then incubated with the secondary antibody for one hour at room temperature. The secondary antibodies included Alexa Fluor® 488 Donkey Anti-Mouse IgG (Invitrogen, A-21202, 1:200), Alexa Fluor® 568 Donkey Anti-Mouse IgG (Invitrogen, A-10037, 1:200), Alexa Fluor® 488 Donkey Anti-Goat IgG (Invitrogen, A-11055, 1:200) and Alexa Fluor® 568 Donkey Anti-Goat IgG (Invitrogen, A-11057, 1:200). After wash three times by cold PBS, the sections were counterstained by DAPI (Cell Signaling, 4083, 1:5000) for five minutes and sealed by coverslips with mounting medium (Sigma-Aldrich, F4680). Finally, fluorescent images were captured by using a Leica confocal or fluorescence microscopy and further analyzed with ImageJ software.

### Administration of Lipopolysaccharide (LPS) and/or MK-8931 to Animals

All animal experiments were performed in accordance with protocols approved by the IACUC. C57BL/6 mice used for the in vivo experiments were maintained in a 14 hours light/10 hours dark cycle, and provided with sterilized water and food ad libitum. LPS was prepared and intraperitoneally injected into adult C57BL/6 mice as reported (45). MK-8931 was dissolved in 0.5% methylcellulose as previously reported (46) and orally administered into mice at the dose of 50 mg/kg. For mouse plasma cytokine assay, mice were given by a single dose of LPS (10 mg/kg) in combination with or without MK-8931 treatment. Twenty-four hours later, mouse plasma were harvested for the cytokine array. For the survival rescue experiment, mice were given by a single lethal dose of LPS (15 mg/kg) in combination with or without MK-8931 (50 mg/kg), and then monitored to record the survival time.

### Collection of Mouse Blood and Plasma Cytokine Assay

Mouse blood samples were withdrawn by cardiac puncture according to an established protocol (47). To prepare plasma, mouse blood was collected into the EDTA-coated Eppendorf tubes. After adding the sterile EDTA to a 5 mM final concentration, the blood was centrifuged for 15 minutes at 3000 rpm at 4° C. The supernatant (plasma) was carefully harvested and mouse cytokine assay was performed by using the Proteome Profiler Mouse Cytokine Array Kit (R&D, ARY006) according to manufacturer's instruction. Briefly, mouse plasma was mixed with a cocktail of biotinylated mouse cytokine detection antibodies and incubated at room temperature for 1 hour. Then, the mixtures of cytokines and detected antibodies were incubated with the Mouse Cytokine Array membrane at 4° C. overnight. The membranes were washed three times with buffer and then incubated with the diluted Streptavidin-HRP buffer for 30 minutes at room temperature. After washing, immunoreactivity was visualized by using the chemiluminescence reagent mix and the signals were acquired and analyzed by Image Lab software (Bio-Rad). The intensity was measured and quantified by using image J software (NIH, Bethesda, MD, USA).

### Hematoxylin-Eosin (H&E) Staining

Mouse tissues were fixed with 4% PFA, cryopreserved in 30% sucrose and snap-frozen in OCT compound (Sakura® Finetek, 4583). Hematoxylin and eosin staining was performed to detect tissue damage. The sections were dehydrated with different concentrations of ethanol and xylene and sealed with neutral gum, and the pathological changes were observed under an optical microscope. According to the previous studies (48-50), tissue damage was scored on the H&E stained sections by using the grades from 0 to 4 as follows: 0, no damage; 1; mild damage; 2, moderate damage; 3, severe damage; and 4, very severe damage and histological changes.

### Statistical Analysis

All bar graphs represent mean±SEM unless otherwise indicated. For the survival analysis, the log-rank survival analysis was performed with GraphPad Prism 5 software to determine significance among groups. Experimental details such as number of animals or cells and experimental replication were provided in the figure legends. Data inclusion/exclusion criteria was not applied in this study. Significant differences were determined between two groups using the Student's t test and statistical significance was set at p<0.05.

### Results

### Spike Protein of SARS-CoV-2 Activates Macrophages to Release Cytokines and Chemokines

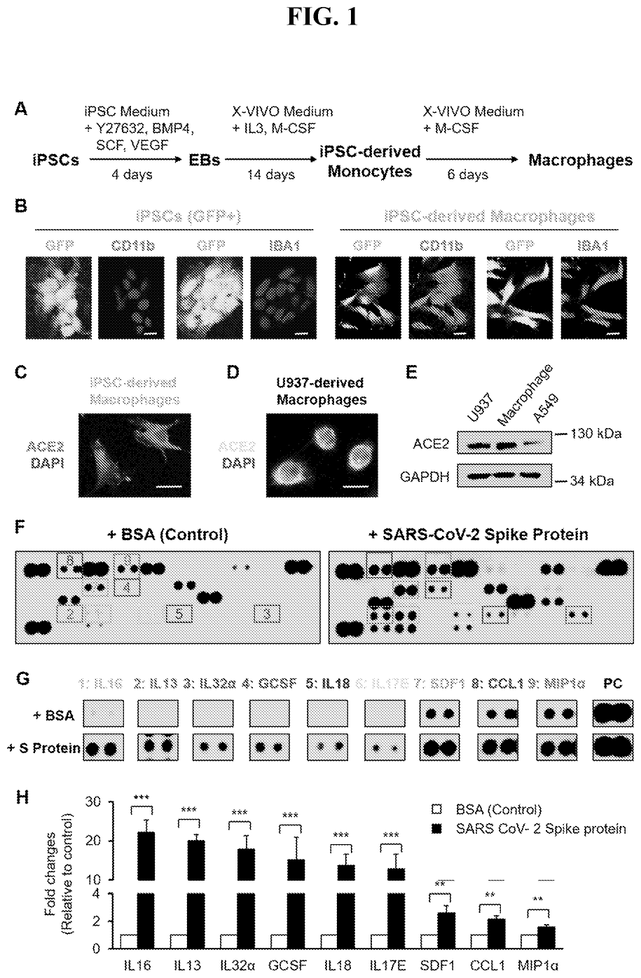

It has been well recognized that the infiltrated monocytes and macrophages in the lung secrete abundant pro-inflammatory cytokines to cause hyper-inflammation, vascular damage, blood clotting, lung injury, and ARDS. To understand how SARS-CoV-2 activates macrophages to produce excessive cytokines/chemokines, we initially examined macrophage response to the stimulation of the SARS-CoV-2 Spike protein, a key protein mediating the binding of coronavirus to ACE2 receptor on human cells including macrophages. We used both U937 monocyte-derived macrophages and the iPS cell-derived macrophages (FIG. 1A) for this study. The iPSC-derived macrophages express typical macrophage markers including IBA1 and CD11b (FIG. 1B). We confirmed that both iPSC-derived macrophages and U937 monocyte-derived macrophages express ACE2 receptor on cell surface as demonstrated by immunofluorescent staining and immunoblot analyses (FIG. 1, C to E). To determine whether the Spike protein of SARS-CoV-2 can activate macrophages to produce and release cytokines and chemokines, we collected conditioned media of macrophages treated with the recombinant Spike protein (rSP) or BSA control for 24 hours to profile key cytokines/chemokines. The cytokine array results showed that several pro-inflammatory cytokines or chemokines (G-CSF, IL-16, IL-13, IL-32a, IL-18, IL-17E, SFD-1, CCL-1 and MIP1α) were markedly induced by the Spike protein in U937 monocyte-derived macrophages (FIG. 1, F to H). Quantifications indicated that most of these cytokines/chemokines in the conditioned medium of macrophages were induced more than 10 folds by the Spike protein (FIG. 1H), indicating that the Spike protein of SARS-CoV-2 is able to induce release of cytokine/chemokines by macrophages. To validate this conclusion, we further used iPSC-derived macrophages to repeat the experiment and obtained similar results. Collectively, these data demonstrate that stimulation with the SARS-CoV-2 Spike protein only is sufficient to activate macrophages to release excessive cytokines/chemokines, indicating that the abundant Spike proteins released from dead coronaviruses in the patient may be potent enough to stimulate macrophages to produce excessive pro-inflammatory cytokines/chemokines, resulting in cytokine storm-related symptoms in severe patients with COVID-19.

### The BACE1 Inhibitor Suppresses the SARS-CoV-2 Spike Protein-Induced Cytokine Release by Macrophages

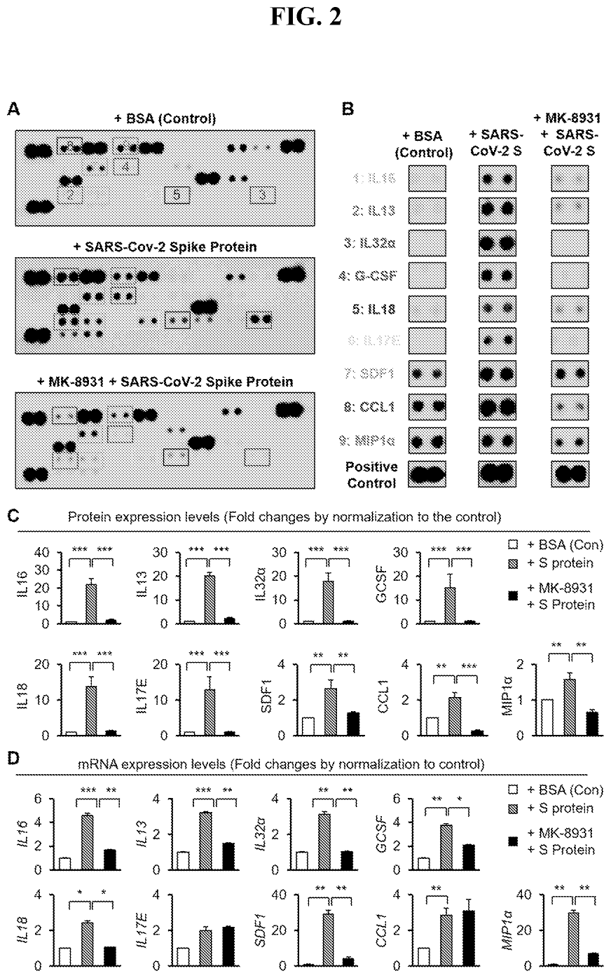

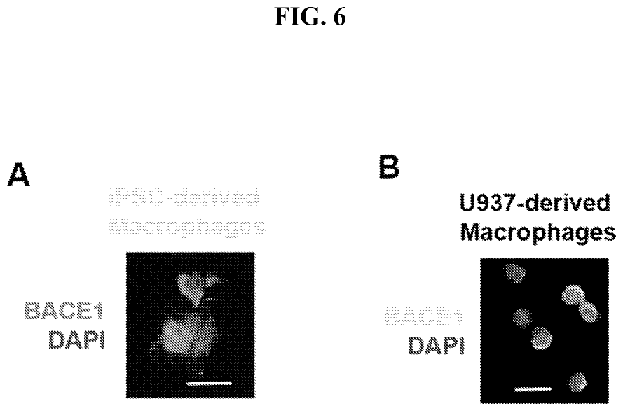

As the Spike protein of SARS-CoV-2 is sufficient to activate macrophages to produce cytokines/chemokines, we sought to identify potential small molecules that can suppress the Spike protein-induced cytokine release by macrophages, while avoiding using dangerous coronaviruses for the drug screening. To discover such new drugs or repurpose existing drugs, we used U937-derived macrophages and iPSC-derived macrophages (FIG. 1, A to E) in combination with cytokine assay to screen an inhibitor library (SelleckChem) and some known drugs in clinical trials for other diseases. To this end, we found that several inhibitors of BACE1 (the (3-site amyloid precursor protein cleaving enzyme 1, aka beta-secretase 1) including MK-8931 originally developed for clinical trials of Alzheimer's disease (AD) were able to suppress the Spike protein-induced release of cytokines/chemokines by macrophages. Cytokine array demonstrated that BACE1 inhibition by MK-8931 abolished the induction of several pro-inflammatory cytokines (G-CSF, IL-16, IL-13, IL-32a, IL-18, IL-17E, SFD-1, CCL-1 and MIP1α) by the Spike protein in macrophages (FIG. 2, A and B). Quantifications indicated that the SARS-CoV-2 Spike protein consistently induced release of several cytokines/chemokines more than 10 fold in macrophages, but co-treatment with MK8931 largely attenuated such induction (FIG. 2C). In addition, RT-PCR analyses confirmed that Spike protein potently induced expression of several cytokines/chemokines, but MK-8931 co-treatment effectively inhibited such induction (FIG. 2D), indicating that the BACE1 inhibitor MK8931 suppresses the SARS-CoV-2 Spike protein-induced expression of pro-inflammatory cytokines in macrophages at transcriptional level. We have validated these results in both U937-derived macrophages and iPSC-derived macrophages. Interestingly, BACE1 is highly expressed by both U937- and iPSC-derived macrophages (FIG. 6). Collectively, these data demonstrate that BACE1 inhibition by MK-8931 effectively suppresses the induction of pro-inflammatory cytokines by SARS-CoV-2 Spike protein in macrophages.

### BACE1 Inhibition by MK-8931 Attenuates the LPS-Induced Cytokine Release by Macrophages

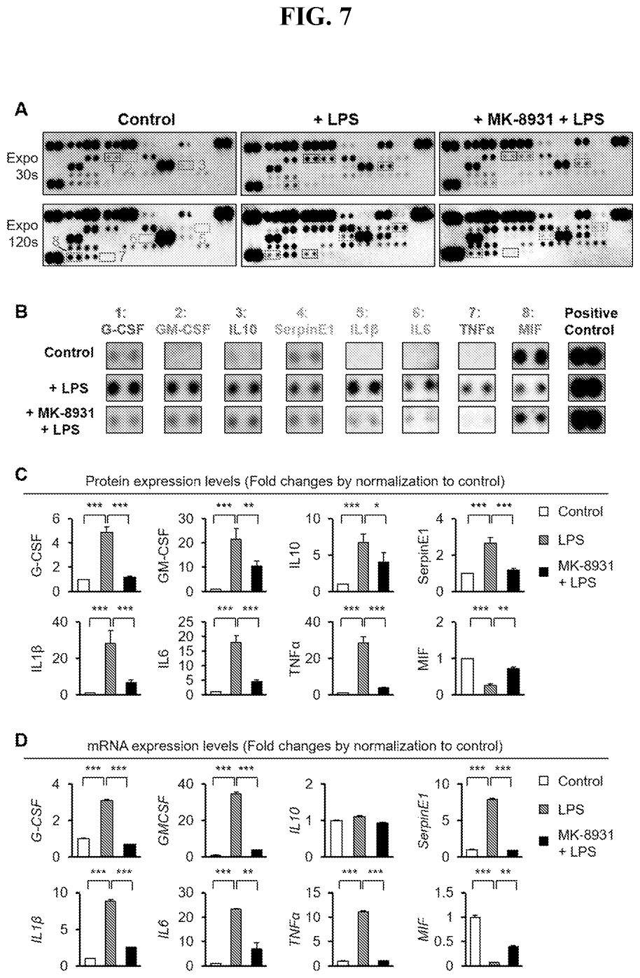

To determine whether the BACE1 inhibitor MK-8931 also suppresses other stimulation-induced cytokine release by macrophages, we examined the effect of MK-8931 treatment on the lipopolysaccharide (LPS)-induced release of pro-inflammatory cytokines/chemokines by macrophages, as LPS is a well-known bacterial agent that can trigger cytokine storm. Cytokine array of conditioned media showed that LPS potently induced secretion of several pro-inflammatory cytokines including IL-6, IL-1β, TNFα, G-CSF, GM-CSF, IL-10, and Serpin E1 by macrophages (FIG. 7, A to C), but MK-8931 co-treatment effectively attenuated such induction of cytokines by LPS in macrophages (FIG. 7, A to C). Moreover, RT-PCR analyses validated that the LPS-induced expressions of these cytokines in macrophages were suppressed by MK-8931 co-treatment (FIG. 7D). Interestingly, we found that LPS stimulation downregulated expression of MIF (Macrophage Migration Inhibitory Factor) by macrophages, but MK-8931 treatment partially rescued the downregulation of MIF (FIG. 7, A to D). Collectively, these data demonstrate that BACE1 inhibition by MK-8931 also potently suppresses the induction of key pro-inflammatory cytokines in macrophages in response to LPS stimulation, indicating that MK-8931 treatment may generally overcome the cytokine storm triggered by varied stimulations including both viral and bacterial infections.

### MK-8931 Treatment Effectively Suppresses the LPS-Induced Cytokine Storm In Vivo

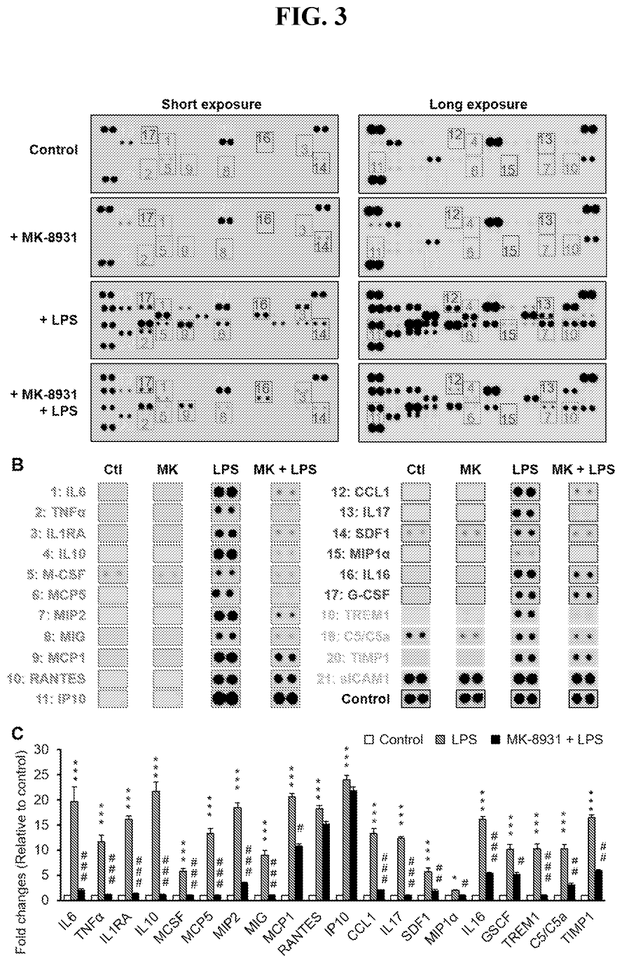

To further determine whether MK-8931 treatment in vivo suppresses the excessive release of pro-inflammatory cytokines induced by LPS, we challenged mice with LPS in combination with or without MK-8931 treatment, and collected serum samples for cytokine profiling. Consistently, cytokine array showed that LPS stimulation induced cytokine storm as indicated by much higher plasma levels of a number of pro-inflammatory cytokines/chemokines (IL-6, TNF-α, IL-1RA, IL-10, M-CSF, MCP5, MIP2, MIG, RANTES, IP10, CCL1, IL-17, SDF-1, MIP1a, IL-16, G-CSF, TREM1, C5/C5a, and TIMP1) in the LPS-treated mice relative to control mice (FIG. 3, A and B). However, MK-8931 co-treatment clearly attenuated such induction of these pro-inflammatory cytokines/chemokine by LPS in vivo (FIG. 3, A and B). Quantifications indicate MK-8931 treatment significantly suppressed the LPS-mediated induction of most pro-inflammatory cytokines /chemokine (FIG. 3C). Among these cytokines/chemokines, IL-6, TNF-α, IL-IRA, IL-10, M-CSF, G-CSF, MCP5, MIP2, MIG, MCP1, RANTES, and IP-10 were found to be commonly increased in sera of COVID-19 patients (2), and CCL1, IL-17, SDF-1, MIP1a, IL-16 and G-CSF were induced directly by stimulation of the SARS-CoV-2 Spike protein (FIGS. 1 and 2). These data suggest that LPS-induced cytokine release largely mimics the SARS-CoV-2-tiggered cytokine storm, but MK-8931 treatment potently overcome such cytokine storm in vivo. Notably, MK-8931 treatment alone did not affect plasma levels of cytokines/chemokines in the mice without LPS treatment (FIG. 3, A and B), which is consistent with the fact that MK-8931 is well-tolerated for patients during AD clinical trials (27, 28). Taken together, these data demonstrate that the treatment with the BACE1 MK-8931 effectively suppresses the cytokine storm in vivo, indicating that MK-8931 could be used to overcome the cytokine storm and relive inflammation-related symptoms in severe patients with COVID-19.

### MK-8931 Treatment Effectively Protected Animals From Cytokine Storm-Induced Death

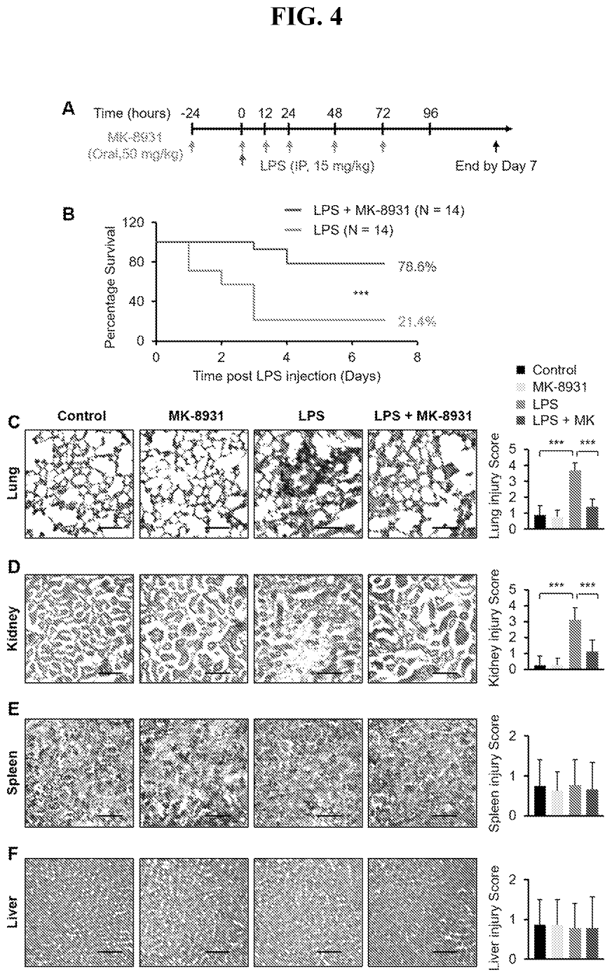

As MK-8931 treatment in vivo potently suppresses the cytokine storm induced by LPS in animals, we next sought to examine whether MK-8931 treatment can prevent or reduce the cytokine storm-induced death. To address this critical issue, we treated one group of mice with a lethal dose of LPS (15 mg/kg) only, and treated another group of mice with MK-8931 (50 mg/kg, oral) and the same dose of LPS, and then monitored animal survival as illustrated (FIG. 4A). In the group of mice treated with the lethal dose of LPS only, 11 of 14 mice (78.6%) died between 20 to 96 hours after LPS administration (FIG. 4B), which is expected. Surprisingly, in the experimental group of mice treated with MK-8931 and LPS, only 3 of 14 mice (21.4%) died between 20 to 96 hours after the LPS administration, and 11 of the 14 mice (78.6%) survived well (FIG. 4B). Remarkably, MK-8931 treatment reduced the mortality rate by 3.7 fold and increased the survival by 367%, demonstrating that MK-treatment potently protects animals from the cytokine storm-induced death caused by the lethal dose of LPS. In addition, histochemical analyses of major organs further confirmed that MK-8931 treatment significantly reduced the inflammation-induced tissue damages in lungs and kidneys in survived animals treated with LPS plus MK-8931 relative to animals treated with LPS only (FIG. 4, C and D), while LPS-induced tissue injury was hardly detected in the spleen and liver (FIG. 4, E and F). The effect of MK-8931 treatment on protecting lungs from the cytokine storm-induced injury is particularly obvious (FIG. 4C), which is important, as lung tissue damages caused by cytokine storm-induced inflammation are often seen in severe patients with COVID-19. Collectively, our data indicate that the BACE1 inhibitor MK-8931 effectively prevents the cytokine storm-related death.

It has been well recognized that most deaths from COVID-19 are mainly caused by the SARS-CoV-2-induced cytokine storm that triggers hyper-inflammation, organ damage and severe symptoms including ARDS. Thus, overcoming cytokine storm by MK-8931 treatment may save many lives of severe patients with COVID-19. As MK-8931 is a safe oral drug and well-tolerated in patients, which has been demonstrated in AD clinical trials (27, 28), repurposing MK-8931 for treating severe COVID-19 patients with cytokine release syndrome should be useful. Our findings indicate that BACE1 inhibitors including MK-8931 have great potential to effectively suppress cytokine storm, reduce inflammation, and relieve severe symptoms including ARDS to improve the survival of severe patients with COVID-19. Clinical trials using a BACE1 inhibitor such as MK-8931 alone or in combination with antiviral therapy or other supportive treatments will demonstrate a promising therapeutic potential to reduce the mortality of COVID-19.

As BACE1 is a transmembrane β-secretase that plays a critical role in the cleavage of amyloid precursor protein to cause accumulation of AP in brains of patients with Alzheimer's disease (AD) (29-32). BACE1 was originally identified as a therapeutic target for AD. Because BACE1 deficiency is well-tolerated in the knockout mice without effects on the development, behavior, and fertility (33), targeting BACE1 should not result in significant side effects. Several BACE1 inhibitors have been developed for clinical trials to treat ADs. These BACE1 inhibitors including MK-8931, AZD3293, E2609, and CNP50 have been shown to be well-tolerated for patients in the AD clinical trials (27, 28, 34, 35), although most of these trials failed due to ineffectiveness for AD control. Thus, repurposing these BACE1 inhibitors for treatment of severe patients with COVID-19 should be safe. BACE1 inhibitor have tremendous therapeutic potential to rescue severe patients with COVID-19. Our findings offer a rapid therapeutic approach to address the urgent need at this critical moment of the COVID-19 crisis.

Because cytokine storm or cytokine release syndrome (CRS) can be triggered by infection of other viruses such as SARS, MERS, influenza and Ebola (2, 9-11), overcoming cytokine storm by BACE1 inhibitors may be used in future pandemics involving in such viruses or other potentially unknown viruses. In addition, as cytokine storm occurs in cancer patients receiving CAR-T cell immunotherapy, in patients with organ transplantation, and in some patients with arthritis or other disorders of immune response, BACE1 inhibitors can be used to suppress the excessive cytokine release in these patients to improve the treatment. Furthermore, because the BACE1 inhibitor simultaneously suppresses the excessive release of several key pro-inflammatory cytokines, and the cost of a small molecular inhibitor will be much less than that using several antibodies against multiple pro-inflammatory cytokines, overcoming cytokine storm with a small molecule inhibitor of BACE1 such as MK-8931 will provide a much more effective and economic therapeutic approach to improve survival of severe patients with COVID-19.

### REFERENCES

1. Z. Wu, J. M. McGoogan, Characteristics of and Important Lessons From the Coronavirus Disease 2019 (COVID-19) Outbreak in China: Summary of a Report of 72314 Cases From the Chinese Center for Disease Control and Prevention. JAMA, (2020).
2. C. Huang et al., Clinical features of patients infected with 2019 novel coronavirus in Wuhan, China. Lancet 395, 497-506 (2020).
3. C. Wu et al., Risk Factors Associated With Acute Respiratory Distress Syndrome and Death in Patients With Coronavirus Disease 2019 Pneumonia in Wuhan, China. JAMA Intern Med, (2020).
4. N. Chen et al., Epidemiological and clinical characteristics of 99 cases of 2019 novel coronavirus pneumonia in Wuhan, China: a descriptive study. Lancet 395, 507-513 (2020).
5. J. Helms et al., High risk of thrombosis in patients with severe SARS-CoV-2 infection: a multicenter prospective cohort study. Intensive Care Med 46, 1089-1098 (2020).
6. F. A. Klok et al., Confirmation of the high cumulative incidence of thrombotic complications in critically ill ICU patients with COVID-19: An updated analysis. Thromb Res 191, 148-150 (2020).
7. F. A. Klok et al., Incidence of thrombotic complications in critically ill ICU patients with COVID-19. Thromb Res 191, 145-147 (2020).
8. M. Ackermann et al., Pulmonary Vascular Endothelialitis, Thrombosis, and Angiogenesis in Covid-19. N Engl J Med, (2020).
9. J. B. Moore, C. H. June, Cytokine release syndrome in severe COVID-19. Science 368, 473-474 (2020).
10. P. Mehta et al., COVID-19: consider cytokine storm syndromes and immunosuppression. Lancet 395, 1033-1034 (2020).
11. Z. Xu et al., Pathological findings of COVID-19 associated with acute respiratory distress syndrome. Lancet Respir Med 8, 420-422 (2020).
12. D. T. Teachey et al., Identification of Predictive Biomarkers for Cytokine Release Syndrome after Chimeric Antigen Receptor T-cell Therapy for Acute Lymphoblastic Leukemia. Cancer Discov 6, 664-679 (2016).
13. C. K. Wong et al., Plasma inflammatory cytokines and chemokines in severe acute respiratory syndrome. Clin Exp Immunol 136, 95-103 (2004).
14. R. Channappanavar, S. Perlman, Pathogenic human coronavirus infections: causes and consequences of cytokine storm and immunopathology. Semin Immunopathol 39, 529-539 (2017).
15. J. R. Tisoncik et al., Into the eye of the cytokine storm. Microbiol Mol Biol Rev 76, 16-32 (2012).
16. B. G. Chousterman, F. K. Swirski, G. F. Weber, Cytokine storm and sepsis disease pathogenesis. Semin Immunopathol 39, 517-528 (2017).
17. A. Shimabukuro-Vomhagen et al., Cytokine release syndrome. J Immunother Cancer 6, 56 (2018).
18. C. Skevaki, P. C. Fragkou, C. Cheng, M. Xie, H. Renz, Laboratory characteristics of patients infected with the novel SARS-CoV-2 virus. J Infect, (2020).
19. H. B. Cohen, D. M. Mosser, Extrinsic and intrinsic control of macrophage inflammatory responses. J Leukoc Biol 94, 913-919 (2013).
20. G. Arango Duque, A. Descoteaux, Macrophage cytokines: involvement in immunity and infectious diseases. Front Immunol 5, 491 (2014).
21. M. Merad, J. C. Martin, Pathological inflammation in patients with COVID-19: a key role for monocytes and macrophages. Nat Rev Immunol 20, 355-362 (2020).
22. M. Liao et al., Single-cell landscape of bronchoalveolar immune cells in patients with COVID-19. Nat Med 26, 842-844 (2020).
23. M. Hoffmann et al., SARS-CoV-2 Cell Entry Depends on ACE2 and TMPRSS2 and Is Blocked by a Clinically Proven Protease Inhibitor. Cell 181, 271-280 e278 (2020).
24. N. Vabret et al., Immunology of COVID-19: Current State of the Science. Immunity 52, 910-941 (2020).
25. S. Keidar et al., Mineralocorticoid receptor blocker increases angiotensin-converting enzyme 2 activity in congestive heart failure patients. Circ Res 97, 946-953 (2005).
26. N. Inandiklioglu, T. Akkoc, Immune Responses to SARS-CoV, MERS-CoV and SARS-CoV-2. Adv Exp Med Biol, (2020).
27. M. F. Egan et al., Randomized Trial of Verubecestat for Mild-to-Moderate Alzheimer's Disease. New England Journal of Medicine 378, 1691-1703 (2018).
28. M. F. Egan et al., Randomized Trial of Verubecestat for Prodromal Alzheimer's Disease. N Engl J Med 380, 1408-1420 (2019).
29. R. Vassar et al., beta-secretase cleavage of Alzheimer's amyloid precursor protein by the transmembrane aspartic protease BACE. Science 286, 735-741 (1999).
30. R. Q. Yan et al., Membrane-anchored aspartyl protease with Alzheimer's disease beta-secretase activity. Nature 402, 533-537 (1999).
31. S. Sinha et al., Purification and cloning of amyloid precursor protein beta-secretase from human brain. Nature 402, 537-540 (1999).
32. I. Hussain et al., Identification of a novel aspartic protease (Asp2) as beta-secretase. Molecular and Cellular Neuroscience 14, 419-427 (1999).
33. R. Yan, Physiological Functions of the beta-Site Amyloid Precursor Protein Cleaving Enzyme 1 and 2. Front Mol Neurosci 10, 97 (2017).
34. J. R. Sims et al., Development Review of the BACE1 Inhibitor Lanabecestat (AZD3293/LY3314814). J Prey Alzheimers Dis 4, 247-254 (2017).
35. L. K. Huang, S. P. Chao, C. J. Hu, Clinical trials of new drugs for Alzheimer disease. J Biomed Sci 27, 18 (2020).
36. B. van Wilgenburg, C. Browne, J. Vowles, S. A. Cowley, Efficient, long term production of monocyte-derived macrophages from human pluripotent stem cells under partly-defined and fully-defined conditions. PLoS One 8, e71098 (2013).
37. S. Mia, A. Warnecke, X. M. Zhang, V. Malmstrom, R. A. Harris, An optimized Protocol for Human M2 Macrophages using M-CSF and IL-4/IL-10/TGF-beta Yields a Dominant Immunosuppressive Phenotype. Scandinavian Journal of Immunology 79, 305-314 (2014).
38. Y. Shi et al., Tumour-associated macrophages secrete pleiotrophin to promote PTPRZ1 signalling in glioblastoma stem cells for tumour growth. Nat Commun 8, 15080 (2017).
39. S. Bao et al., Glioma stem cells promote radioresistance by preferential activation of the DNA damage response. Nature 444, 756-760 (2006).
40. L. Cheng et al., Glioblastoma stem cells generate vascular pericytes to support vessel function and tumor growth. Cell 153, 139-152 (2013).
41. X. Fang et al., The zinc finger transcription factor ZFX is required for maintaining the tumorigenic potential of glioblastoma stem cells. Stem Cells 32, 2033-2047 (2014).
42. X. Fang et al., Deubiquitinase USP13 maintains glioblastoma stem cells by antagonizing FBXL14-mediated Myc ubiquitination. J Exp Med 214, 245-267 (2017).
43. Y. Shi et al., Tetraspanin CD9 stabilizes gp130 by preventing its ubiquitin-dependent lysosomal degradation to promote STAT3 activation in glioma stem cells. Cell Death Differ 24, 167-180 (2017).
44. W. Zhou et al., Periostin secreted by glioblastoma stem cells recruits M2 tumour-associated macrophages and promotes malignant growth. Nat Cell Biol 17, 170-182 (2015).
45. K. Raduolovic, R. Mak'Anyengo, B. Kaya, A. Steinert, J. H. Niess, Injections of Lipopolysaccharide into Mice to Mimic Entrance of Microbial-derived Products After Intestinal Barrier Breach. J Vis Exp, (2018).
46. M. E. Kennedy et al., The BACE1 inhibitor verubecestat (MK-8931) reduces CNS beta-amyloid in animal models and in Alzheimer's disease patients. Science Translational Medicine 8, (2016).
47. S. Parasuraman, R. Raveendran, R. Kesavan, Blood sample collection in small laboratory animals. J Pharmacol Pharmacother 1, 87-93 (2010).
48. T. Zhu et al., Andrographolide protects against LPS-induced acute lung injury by inactivation of NF-kappaB. PLoS One 8, e56407 (2013).
49. I. N. Baranova et al., Human SR-BI and SR-BII Potentiate Lipopolysaccharide-Induced Inflammation and Acute Liver and Kidney Injury in Mice. J Immunol 196, 3135-3147 (2016).
50. Z. Chen, H. Liu, S. Lei, B. Zhao, Z. Xia, LY294002 prevents lipopolysaccharide induced hepatitis in a murine model by suppressing IkappaB phosphorylation. Mol Med Rep13, 811-816 (2016).
51. US Pat. Pub. 20040229816.
52. KR100881747.
53. Macleod et al., Future Sci OA. 2015 November; 1 (3): FSO11.
54. Rizzo et al., Basic Res Cardiol. 2020; 115 (3): 31.
55. Woodard-Grice et al., J Biol Chem. 2008 Sep. 26; 283 (39): 26364-26373.
56. Lichtenthaler et al., J Biol Chem. 278 (49):48713-9.
57. Moussa-Pacha et al., Med Res Rev. 2020; 40:339-384.
58. Wessels et al., JAMA Neurol. 2020; 77 (2):199-209.

All publications and patents mentioned in the present application are herein incorporated by reference. Various modification and variation of the described methods and compositions of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

We claim:

1. A method of treating a subject with a condition that causes a cytokine storm in the subject comprising:
administering a composition to a subject, or providing said composition to said subject such that said subject administers said composition to themselves,
wherein said subject has a condition that causes a cytokine storm, and
wherein said composition comprises a beta-secretase 1 (BACE1) inhibitor.

2. The method of claim 1, wherein said condition is infection by a virus that causes said cytokine storm.

3. The method of claim 2, wherein said virus is SARS-CoV-2 (COVID-19).

4. The method of claim 2, wherein said virus is a coronavirus.

5. The method of claim 2, wherein said virus is selected from the group consisting of: MERS, SARS-COV-1, RSV, adenovirus, influenza, and Ebola.

6. The method of claim 1, wherein said condition is: having received CAR-T cell immunotherapy, having receive an organ transplant, and/or having an autoimmune disease.

7. The method of claim 1, wherein said autoimmune disease is arthritis.

8. The method of claim 1, wherein said providing comprises giving said composition to said subject in the form of oral pills that said patient takes themselves.

9. The method of claim 1, wherein said administering comprises injecting said composition into said subject.

10. The method of claim 1, further comprising: c) repeating said administering or providing daily for at least one week or at least three weeks.

11. The method of claim 1, wherein said administering comprises administering 0.05 mg of said BACE1 inhibitor per kg of the subject to 50 mg per kg of the subject, or administering a total dose of 3-1000 mg of said BACE1 inhibitor.

12. The method of claim 1, wherein said BACE1 inhibitor comprises MK-8931 (Verubecestat).

13. The method of claim 1, wherein said BACE1 inhibitor is selected from the group consisting of: AZD3293 (Lanabecestat), E2609 (Elenbecestat), CNP50, CNP2609, PF-06751979, and JNJ-54861911 (Atabecestat).

14. The method of claim 1, wherein said BACE1 inhibitor is selected from Table 1.

15. The method of claim 1, wherein said subject is a human.

16. The method of claim 1, wherein said administering is such that said subject receives about 0.5-4.0 mg of said BACE1 inhibitor per kilogram of said patient 1-5 times per day for at least 1 day.

17. The method of claim 1, wherein said administering comprises intravenous administration.

18. The method of claim 1, wherein said administering is via said subject's airway.

19. A composition comprising:
a) a physiologically tolerable buffer;
b) a BACE1 inhibitor; and
c) an anti-inflammatory agent and/or an agent that reduces SARS-CoV-2 replication or infection rate in vivo.

20. A kit or system comprising:
a) a BACE1 inhibitor; and
b) an anti-inflammatory agent and/or an agent that reduces SARS-CoV-2 replication or infection rate in vivo.


SEQUENCE LISTING (20 sequences)

SEQ ID NO: 1 - DNA, 21 bp, Artificial sequence
  Synthetic primer
aatcatggaggaggatgcctt

SEQ ID NO: 2 - DNA, 20 bp, Artificial sequence
  Synthetic primer
gtcacagcggagatagtgcc

SEQ ID NO: 3 - DNA, 20 bp, Artificial sequence
  Synthetic primer
gcatgtgaatgccatccagg

SEQ ID NO: 4 - DNA, 20 bp, Artificial sequence
  Synthetic primer
cacaggaagtttccggggtt

SEQ ID NO: 5 - DNA, 20 bp, Artificial sequence
  Synthetic primer
agagttgcaacctcagcctc

SEQ ID NO: 6 - DNA, 20 bp, Artificial sequence
  Synthetic primer
aacaacttgggctggtcaca

SEQ ID NO: 7 - DNA, 20 bp, Artificial sequence
  Synthetic primer
gcctaacatgcttcgagatc

SEQ ID NO: 8 - DNA, 20 bp, Artificial sequence
  Synthetic primer
tgatgtctgggtcttggttc

SEQ ID NO: 9 - DNA, 20 bp, Artificial sequence
  Synthetic primer
ggagaaacccagcagcagat

SEQ ID NO: 10 - DNA, 20 bp, Artificial sequence
  Synthetic primer
ccggaacagcctgaagaagt

SEQ ID NO: 11 - DNA, 20 bp, Artificial sequence
  Synthetic primer
tttgagtctgcccagttccc

SEQ ID NO: 12 - DNA, 20 bp, Artificial sequence
  Synthetic primer
tcagttatatcctggccgcc

SEQ ID NO: 13 - DNA, 24 bp, Artificial sequence
  Synthetic primer
tgacaaacaaattcggtacatcct

SEQ ID NO: 14 - DNA, 22 bp, Artificial sequence
  Synthetic primer
agtgcctctttgctgctttcac

SEQ ID NO: 15 - DNA, 20 bp, Artificial sequence
  Synthetic primer
gagcactgaaagcatgatcc

SEQ ID NO: 16 - DNA, 21 bp, Artificial sequence
  Synthetic primer
cgagaagatgatctgactgcc

SEQ ID NO: 17 - DNA, 20 bp, Artificial sequence
  Synthetic primer
agaaccgctcctacagcaag

SEQ ID NO: 18 - DNA, 20 bp, Artificial sequence
  Synthetic primer
gcgaaggtggagttgttcca

SEQ ID NO: 19 - DNA, 21 bp, Artificial sequence
  Synthetic primer
aaggtgaaggtcggagtcaac

SEQ ID NO: 20 - DNA, 22 bp, Artificial sequence
  Synthetic primer
ggggtcattgatggcaacaata